(19) United States
(12) Patent Application Publication
(10) Pub. No.: US 2002/0156063 A1
(43) Pub. Date: Oct. 24, 2002

(54) Arylacetamido-ketobenzoxazole as cysteine protease inhibitors

(76) Inventors: Eduardo L. Setti, San Mateo, CA (US); Shankar Venkatraman, San Diego, CA (US)

Correspondence Address: AXYS PHARMACEUTICALS, INC., 180 KIMBALL WAY, SOUTH SAN FRANCISCO, CA, 94080, US

(21) Appl. No.: 10/118,665

(22) Filed: Apr. 8, 2002

Related U.S. Application Data
(60) Provisional application No. 60/281,932, filed on Apr. 6, 2001.

(51) Int. Cl. A61K 31/397; A61K 31/55; A61K 31/445; A61K 31/4015; A61K 31/401; A61K 31/16

(52) U.S. Cl. 514/210.17; 514/423; 514/217.11; 514/330; 514/625; 514/626; 540/607; 548/950; 548/530; 546/225

(57) ABSTRACT

The present invention thus provides compounds of Formula I:

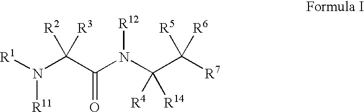

pharmaceutical compositions comprising compounds (s) of Formula I and methods of treating diseases associated with cysteine protease activity using pharmaceutical compositions comprising compounds of Formula I.

### CROSS REFERENCE

This application claims priority under 35 U.S.C. 119(e) to U.S. Provisional Application Serial No. 60/281,932, filed on Apr. 6, 2001, the disclosure of which is incorporated herein by reference in its entirety.

### BACKGROUND OF INVENTION

1. Field of Invention

This invention relates to compounds and compositions for treating diseases associated with cysteine protease activity, particularly diseases associated with activity of cathepsin K.

2. State of the Art

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increased expression or enhanced activation, however, may have pathological consequences.

In this regard, certain cysteine proteases are associated with a number of disease states, including arthritis, muscular dystrophy, inflammation, tumor invasion, glomerulonephritis, malaria, periodontal disease, metachromatic leukodystrophy and others. For example, the prominent expression of cathepsin K in osteoclasts and osteoclast-related multinucleated cells and its high collagenolytic activity suggest that the enzyme is involved in osteoclast-mediated bone resorption and, hence, in bone abnormalities such as occurs in osteoporosis. In addition, cathepsin K expression in the lung and its elastinolytic activity suggest that the enzyme plays a role in pulmonary disorders as well.

In view of the number of diseases wherein it is recognized that an increase in cysteine protease activity contributes to the pathology and/or symptomatology of the disease, molecules which are shown to inhibit the activity of this class of enzymes, in particular molecules which are inhibitors of cathepsin K will be useful as therapeutic agents.

### SUMMARY OF INVENTION

In one aspect, this invention is directed to a compound of Formula I:

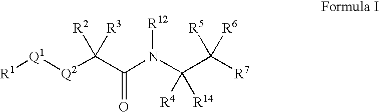

wherein:

R¹ represents —(CH₂)₀₋₂-Z¹-X¹;

Q¹—(CH₂)₀₋₁;

Q² represents a bond, S(O)₂, O or NR¹¹;

R² represents H;

R³ represents H or —C₃₋₆ alkyl; alternatively

R² and R³ along with the carbon atom to which they are attached form a three to eight membered cycloalkylene or heterocycloalkylene moiety;

R⁴ represents —C₁₋₈ alkyl, —(CH₂)₁₋₆-aryl, —(CH₂)₁₋₆—S(O)ₙ—C₁₋₄-alkyl, —(CH₂)₁₋₆-cycloalkyl, —(CH₂)₁₋₆—S(O)ₙ-cycloalkyl, —(CH₂)₁₋₆—S(O)ₙ—C₁₋₄-aryl, —(CH₂)₁₋₆-heteroaryl, —(CH₂)₁₋₆—S(O)ₙ—C₁₋₄-heteroaryl, —(CH₂)₁₋₆-heterocycloalkyl, —(CH₂)₁₋₆—NR^{a}R^{b} (where R^{a} and R^{b} are independently hydrogen, —C₁₋₆ alkyl, or —C(═NH)—NH₂), or —(CH₂)₁₋₆—S(O)ₙ—C₁₋₄-heterocycloalkyl;

R¹⁴ represents H; alternatively

R⁴ and R¹⁴ along with the carbon atom to which they are attached form a three to eight membered cycloalkylene or heterocycloalkylene moiety;

R⁵ represents H;

R⁶ represents —OH; alternatively

R⁵ and R⁶ along with the carbon atom to which they are attached represent a —CO— group;

R⁷ represents a five to ten membered heteroaryl or a five to ten membered heterocycloalkyl moiety, said moieties substituted with R^{8,} R⁹, and R¹⁰;

R¹¹ represents H, —C₁₋₆-alkyl, —C₁₋₆ alkyl substituted with hydroxy, alkoxy, or 2-hydroxyethyl; alternatively

R¹¹ and R² along with the nitrogen and carbon atoms to R¹¹ and R² are respectively attached can be taken together to form a 5-6 membered heterocycloalkylene moiety optionally containing one to three additional heteroatoms selected from nitrogen, oxygen and sulfur;

R¹² represents H or —C₁₋₆-alkyl;

Z¹ represents arylene, heteroarylene, cycloalkylene or heterocycloalkylene;

X¹ represents H or —(CH₂)₀₋₃—L—(CH₂)₀₋₃—Z²13 X²;

n represents an integer from zero to two; and

R⁸, R⁹ and R¹⁰ independently represent H, alkyl, cycloalkyl, halogen, nitro, cyano, —OH, —OR¹⁷, —S(O)ₙ-alkyl, —NH-alkyl, —N(alkyl)₂, —COOR¹⁷, —COOH, —SO₂N(R¹⁸)₂, —SO₂NR ¹⁷R¹⁸, —CON(R¹⁸)₂, —CONR¹⁷R¹⁸, —CONR¹⁸COOH, —CONR¹⁸COOR¹⁷, aryl, a five to ten membered heteroaryl or a five to ten membered heterocycloalkyl moiety;

Z² represents arylene, heteroarylene, cycloalkylene or heterocycloalkylene;

X² represents H or —(CH₂)₀₋₃—L—(CH₂)⁰⁻³—Z³—X³;

R¹⁷ represents H or —(C₁₋₆)alkyl, wherein said —(C₁₋₆)alkyl is substituted with zero to four substituents selected from halogen, —OR³², —SR³², —S(O)R³², —S(O)₂R³², —C(O)R³², —C(O)OR³², —NR³²R³³, —NR³³C(O)OR³², —C(O)NR³²R³³, —S(O)₂NR³²R³³, —NR³³C(O)NR³²R³³, —NR³³C(NR³³)NR³²R³³, —(CH₂)₀₋₆-cycloalkyl, —(CH₂)₀₋₆-diphenyl, —(CH₂)₀₋₆-heterocycloalkyl, —(CH₂)₀₋₆-aryl, —(CH₂)₀₋₆-heteroaryl or —(CH₂)₀₋₆-diheteroaryl;

R¹⁸ represents aryl, H, —C₁₋₆ alkyl, cycloalkyl or heteroaryl;

Z³ represents arylene, heteroarylene, cycloalkylene or heterocycloalkylene;

X³ represents H or —(CH₂)₀₋₃—L—(CH₂)₀₋₃—Z⁴;

L represents a bond, —S(O)ₙ—, —COO—, —O—CO—, —NR³⁵13 SO₂—, —CO—NR³⁵—SO₂NR³⁵—, —NR³⁵—CO—, —CO—, —NR³⁵—COO—, —COONR³⁵—, —NR³⁵—, —O—, or NR³⁵—C(NR³⁵)—;

R³² represents hydrogen, —C₁₋₆-alkyl, —(CH₂)₀₋₃-cycloalkyl, —(CH₂)₀₋₆-heterocycloalkyl, —(CH₂)₀₋₆-aryl, (CH₂)₀₋₆-diphenyl, —(CH₂)₀₋₆-heteroaryl, or —(CH₂)₀₋₆-diheteroaryl;

each R³³ represents hydrogen, —C₁₋₆-alkyl, —(CH₂)₀₋₃-cycloalkyl, —(CH₂)₀₋₆-heterocycloalkyl, —(CH₂)₀₋₆-aryl, or —(CH₂)₀₋₆-heteroaryl;

each R³⁵ represents H or C₁₋₄ alkyl; and

Z⁴ represents aryl, heteroaryl, cycloalkyl or heterocycloalkyl;

or a pharmaceutically acceptable salt thereof.

Preferably, the compound of Formula I is that wherein:

R¹ represents —(CH₂)₀₋₂—Z¹—X¹;

Q¹ (CH₂)₀₋₁;

Q² represents a bond, S(O)₂, O or NR¹¹;

R² represents H;

R³ represents H or C₃₋₆ alkyl; alternatively

R² and R³ along with the carbon atom to which they are attached form a three to six membered cycloalkylene or heterocycloalkylene moiety;

R⁴ represents —C₁₋₈ alkyl, —(CH₂)₁₋₆-aryl, —(CH₂)₁₋₆—S(O)ₙ—C₁₋₄-alkyl, —(CH₂)₁₋₆-cycloalkyl, —(CH₂)₁₋₆—S(O), —C₁₋₄-aryl, —(CH₂)₁₋₆-heteroaryl, —(CH₂)₁₋₆—S(O)ₙ—C₁₋₄-heteroaryl, —(CH₂)₁₋₆-heterocycloalkyl, or —(CH₂)₁₋₆—S(O)ₙ—C₁₋₄-heterocycloalkyl;

R¹⁴ represents H; alternatively

R⁴ and R¹⁴ along with the carbon atom to which they are attached form a three to six membered cycloalkylene or heterocycloalkylene moiety;

R⁵ represents H;

R⁶ represents —OH; alternatively

R⁵ and R⁶ along with the carbon atom to which they are attached represent a —CO—group;

R⁷ represents a five to ten membered heteroaryl or a five to ten membered heterocycloalkyl moiety, said moieties substituted with R⁸, R⁹, and R¹⁰;

R¹¹ represents H or C₁₋₆-alkyl; alternatively

R¹¹ and R² along with the nitrogen and carbon atoms to R¹¹ and R² are respectively attached can be taken together to form a 5-6 membered ring system containing one to three hetero atoms selected from nitrogen, oxygen and sulfur;

R¹² represents H or C₁₋₆-alkyl;

Z¹ represents arylene, heteroarylene, cycloalkylene or heterocycloalkylene;

X¹ represents H or —(CH₂)₀₋₃—L—(CH₂)₀₋₃—Z²—X²;

n represents an integer from zero to two; and

R^{8,} R⁹ and R¹⁰ independently represent H, alkyl, cycloalkyl, halogen, nitro, cyano, —OH, —OR¹⁷, —S(O)ₙ-alkyl, —NH-alkyl, N(alkyl)₂, —COOR¹⁷, —COOH, —SO₂N(R¹⁸)₂, —SO₂NR¹⁷R¹⁸, —CON(R¹⁸)₂, —CONR¹⁷R¹⁸, —CONR¹⁸COOH, —CONR¹⁸COOR¹⁷;

Z² represents arylene, heteroarylene, cycloalkylene or heterocycloalkylene;

X² represents H or —(CH₂)₀₋₃—L—(CH₂)₀₋₃—Z³—X³;

R¹⁷ represents H or (C₁₋₆)alkyl, wherein said (C₁₋₆)alkyl is substituted with zero to four substituents selected from halogen, —OR³², —SR³², —S(O)R³², —S(O)₂R³², —C(O)R³², —C(O)OR³², —NR³²R³³, —NR³³C(O)OR³², —C(O)NR³²R³³, —S(O)₂NR³²R³³, —NR ³³C(O)NR³²R³³, —NR³³C(NR³³)NR³²R³³, —(CH₂)₀₋₆-cycloalkyl, —(CH₂)₀₋₆-diphenyl, —(CH₂)₀₋₆-heterocycloalkyl, —(CH₂)₀₋₆-aryl, (CH₂)₀₋₆-heteroaryl or —(CH₂)₀₋₆-diheteroaryl;

R¹⁸ represents aryl, H, —C₁₋₆ alkyl, cycloalkyl or heteroaryl;

Z³ represents arylene, heteroarylene, cycloalkylene or heterocycloalkylene;

X³ represents H or —(CH₂)₀₋₃—L—(CH₂)₀₋₃;

L represents a bond, —S(O)ₙ—, —COO—, —O —CO—, —NR³⁵—SO₂—, —CO—NR³⁵—SO₂NR³⁵, —NR³⁵—CO—, —CO—, NR³⁵—COO—, —COONR ³⁵—, —NR³⁵—, —, or —NR³⁵—C(NR³⁵)—; R³²represents hydrogen, —C₁₋₆-alkyl, —(CH₂)₀₋₃-cycloalkyl, —(CH₂)₀₋₆-heterocycloalkyl, —(CH₂)₀₋₆-aryl, —(CH₂)₁₋₆-diphenyl, —(CH₂)₀₋₆-heteroaryl, or —(CH₂)₁₋₆-diheteroaryl;

R³³ represents hydrogen, —C₁₋₆-alkyl, —(CH₂)₀₋₃-cycloalkyl, —(CH₂)₀₋₆-heterocycloalkyl, —(CH₂)₀₋₆-aryl, or —(CH₂)₀₋₆-heteroaryl;

R³⁵ represents H or C₁₋₄ alkyl; and

Z⁴ represents arylene, heteroarylene, cycloalkylene or heterocycloalkylene;

or a pharmaceutically acceptable salt thereof.

In a second aspect, this invention is directed to a pharmaceutical comprising a therapeutically effective amount of a compound of Formula I or a pharmaceutically acceptable salt thereof and a pharmaceutically acceptable excipient. The pharmaceutical composition can also comprise one or more active ingredient(s) selected from the group consisting of (i) a therapeutically effective amount of a bisphosphonic acid or an acid ester thereof or a pharmaceutically acceptable salt thereof and (ii) a therapeutically effective amount of an estrogen receptor agonist or a pharmaceutically acceptable salt thereof.

The bisphosphonic acid(s) used in the composition of the present invention is/are selected from the group consisting of 1,1-dichloromethylene-1,1-diphosphonic acid, 1-hydroxy-3-pyrrolidin-1-ylpropylidene-1,1-bisphosphonic acid, 1-hydroxyethylidene-1,1-diphosphonic acid, 1-hydroxy-3-(N-methyl-N-pentylamino)propylidene-1,1-bisphosphonic acid, 6-amino-1-hydroxyhexylidene-1,1-bisphosphonic acid, 3-(dimethylamino)-1-hydroxypropylidene-1,1-bisphosphonic acid, 3-amino-1-hydroxypropylidene-1,1-bisphosphonic acid, 2-pyrid-2-ylethylidene-1,1-bisphosphonic acid, 1-hydroxy-2-pyrid-3-ylethylidene-1,1-bisphosphonic acid, 4-chlorophenylthiomethylenebisphosphonic acid and 1-hydroxy-2-(1H-imidazol-1-yl)ethylidene-1,1-bisphosphonic acid or acid ester thereof or a pharmaceutically acceptable salt thereof.

Particularly preferred pharmaceutical compositions of the present invention comprise 1,1-dichloromethylene-1,1-diphosphonic acid, 1,1-dichloromethylene-1,1-diphosphonate monosodium trihydrate or a pharmaceutically acceptable salt thereof.

In a third aspect, this invention is directed to a method of treating a disease in an animal in which inhibition of a cysteine protease, in particular cathepsin K, can prevent, inhibit or ameliorate the pathology and/or symptomatology of the disease, which method comprises administering to the animal a pharmaceutical composition comprising a therapeutically effective amount of compound of Formula I, an individual isomer, mixture of isomers or a pharmaceutically acceptable salt thereof. A preferred method is one wherein the disease is osteoporosis and the animal being treated is a human. Particularly preferred method comprises a post-menopausal woman as the animal being treated and wherein the cysteine protease is cathepsin K activity.

The above method can also be carried out by administering to said human a pharmaceutical composition comprising a compounds of Formula I, an individual isomer, mixture of isomers or a pharmaceutically acceptable salt thereof in combination with one or more active ingredient(s) selected from the group consisting of (i) a therapeutically effective amount of a bisphosphonic acid or an acid ester thereof or a pharmaceutically acceptable salt thereof and (ii) a therapeutically effective amount of an estrogen receptor agonist or a pharmaceutically acceptable salt thereof.

### DETAILED DESCRIPTION OF THE INVENTION

Definitions

Unless otherwise specified the following terms used in the claims and specification have the meanings give below:

The terms “alkyl” and “alkylene” as used herein represent a saturated or partially unsaturated straight chain or branched hydrocarbon group having from one (1) to fourteen (14) carbon atoms, unless indicated otherwise. Illustrative examples of an alkyl group are methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like. Illustrative examples of an alkylene group are methylene, ethylene, propylene, isopropylene, butylene, propenylene, butenylene, ethynelene, propynylene, butynylene and the like. An alkyl or alkylene group is substituted with one to four groups selected from hydrogen, phenyl, heteroaryl, halogen, nitro, cyano, —OR¹¹, —C(O)R¹¹, —C(O)OR¹¹, —C(O)NR¹¹R¹¹, —S(O)₂NR¹¹R¹¹, —X⁷NR¹¹R¹¹, —X⁷NR¹¹C(O)OR¹¹, —X⁷NR¹¹C(O)NR¹¹R¹¹ or —X⁷NR¹¹C(NR¹¹NR¹¹R¹¹, wherein X⁷ represents is a bond or —(CH₂)₁₋₆— and each R¹¹ independently is hydrogen or —(C₁₋₆)alkyl. When the alkyl group is substituted with two phenyl or heteroaryl it is referred to as diphenyl or diheteroaryl respectively.

The following representations further illustrate the terms “alkyl” and “alkylene”.

“Alkyl”:

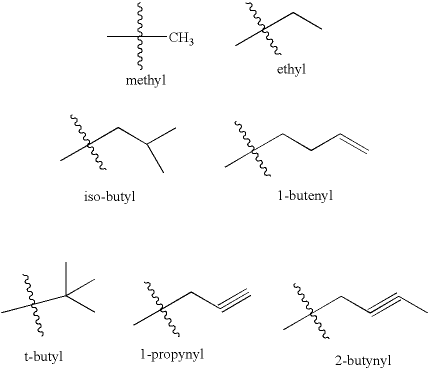

“Alkylene”

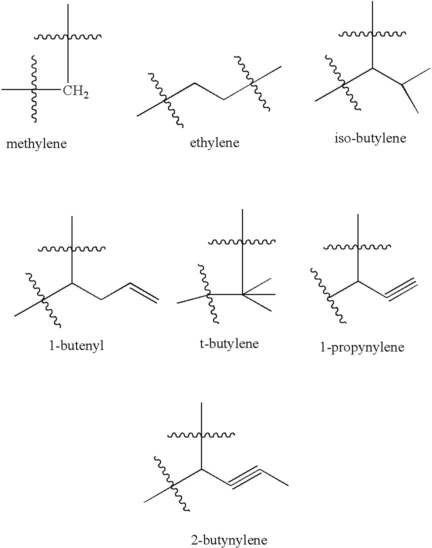

The term “alkoxy” means a radical —OR where R is an alkyl group as defined above, e.g., methoxy, ethoxy, and the like.

The terms “cycloalkyl” and “cycloalkylene” represent a saturated or partially unsaturated, monocyclic, bicyclic or tricyclic ring system having from three (3) to fourteen (14) carbon atoms, unless indicated otherwise. Illustrative examples of a cycloalkyl group are cyclopropyl, cyclobutyl, cyclopentyl, cyclohexyl, cyclohexenyl, 1-methyl-cyclohexyl, 2,5-cyclohexadienyl, bicyclohexylyl, cyclopentylcyclohexyl, bicyclo[2.2.2]octyl, adamantan-1-yl, decahydronaphthalenyl, oxocyclohexyl, dioxocyclohexyl, thiocyclohexyl, 2-oxobicyclo[2.2.1]hept-1-yl, and the like. Illustrative examples of a “cycloalkylene” group are cyclobutylene, cyclohexylene, cyclopropenylene, cyclobutenylene, ethynelene, cyclopropynylene, cyclobutynylene and the like. A cycloalkyl and cycloalkylene ring system is substituted with one to four groups selected from hydrogen, halogen, nitro, cyano, C₁₋₈ straight chain alkyl, C₃₋₁₄ branched alkyl, —OR¹¹, —C(O)R¹¹, —C(O)OR¹¹, —C(O)NR¹¹R¹¹, —S(O)₂NR¹¹R¹¹, —X⁷NR¹¹R¹¹, —X⁷NR¹¹C(O)OR¹¹, —X⁷NR¹¹C(O)NR¹¹R¹¹ or —X⁷NR¹¹C(NR¹¹NR¹¹R¹¹, wherein X⁷ represents is a bond or —(CH₂)₁₋₆— and each R¹¹ independently is hydrogen or —(C₁₋₆)alkyl.

The following representations further illustrate the terms “cycloalkyl” and “cycloalkylene”.

“Cycloalkyl”:

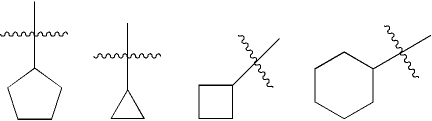

Cycloalkylene:

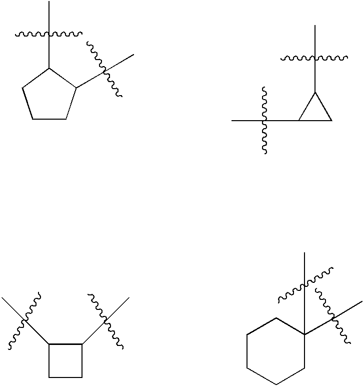

The terms “aryl” or “arylene” as used herein represent a monocyclic, bicyclic, or tricyclic aromatic moiety having from six to fourteen (14) carbon atoms, unless indicated otherwise. The aromatic moiety can be fused a ring (e.g., naphthalene) or a bicyclic ring system wherein two aromatic rings are connected to each other by a bond (e.g., biphenyl). Illustrative examples of a monocyclic aromatic moiety are phenyl, of a fused bicyclic aromatic moiety is naphthyl and of a fused tricyclic aromatic moiety is anthracyl. An aryl or arylene moiety is substituted with one to four groups selected from hydrogen, halogen, nitro, cyano, C₁₋₈ straight chain alkyl, C₃₋₁₄ branched alkyl, —OR¹¹, —C(O)R¹¹, —C(O)OR¹¹, —C(O)NR¹¹R¹¹, —S(O)₂NR¹¹R¹¹, —X⁷NR¹¹R¹¹, —X⁷NR¹¹C(O)OR¹¹, —X⁷NR¹¹C(O)NR¹¹R¹¹ or —X⁷NR¹¹C(NR¹¹NR¹¹R¹¹, wherein X⁷ represents is a bond or —(CH₂)₁₋₆— and each R¹¹ independently is hydrogen or —(C₁₋₆)alkyl.

The following representations further illustrate the terms “aryl” and “arylene”.

Aryl:

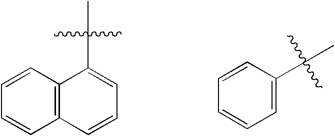

Arylene:

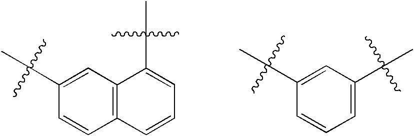

The term “halogen” means fluoro, chloro, bromo, and iodo, preferably fluoro or chloro.

The terms “heteroaryl” and “heteroarylene” represent an “aryl” having 5 to 14 carbon atoms but wherein at least one and maximum of six (6) carbon atoms in an aryl group are replaced with a hetero atom selected from S, O and N. The hetero atoms can exist in their respective oxidized states. Thus a sulfur (S) atom can exist as a sulfoxide or sulfone, while a nitrogen (N) can exist as in the form of an N-oxide group. Illustrative examples of a “heteroaryl” group are thienyl, furyl, pyrrolyl, pyrimidinyl, isoxazolyl, oxaxolyl, indolyl, benzo[b]thienyl, isobenzofuranyl, purinyl, isoquinolyl, pterdinyl, perimidinyl, imidazolyl, 1-methylimidazolyl, 1-benzylimidazolyl, pyridyl, pyrazolyl, pyrazinyl, quinolyl, [2,4′]bipyridinylyl, 2-phenylpyridyl, 4-thiazol-4-ylphenyl, and the like. As in the case of an “aryl” group above, a “heteroaryl” group or moiety is substituted with one to four groups selected from hydrogen, halogen, nitro, cyano, C₁₋₈ straight chain alkyl, C₃₋₁₄ branched alkyl, —OR¹¹, —C(O)R¹¹, —C(O)OR¹¹, —C(O)NR¹¹R¹¹, —S(O)₂NR¹¹R¹¹, —X⁷NR¹¹R¹¹, —X⁷NR¹¹C(O)OR¹¹, —X⁷NR¹¹C(O)NR¹¹R¹¹ or —X⁷NR¹¹C(NR¹¹NR¹¹R¹¹, wherein X⁷ represents is a bond or —(CH₂)₁₋₆— and each R¹¹ independently is hydrogen or —(C₁₋₆)alkyl. The following representations further illustrate the terms “heteroaryl” and “heteroarylene”.

Heteroaryl:

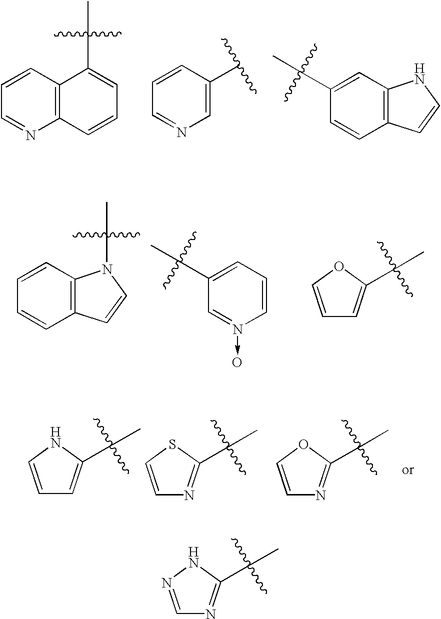

Heteroarylene:

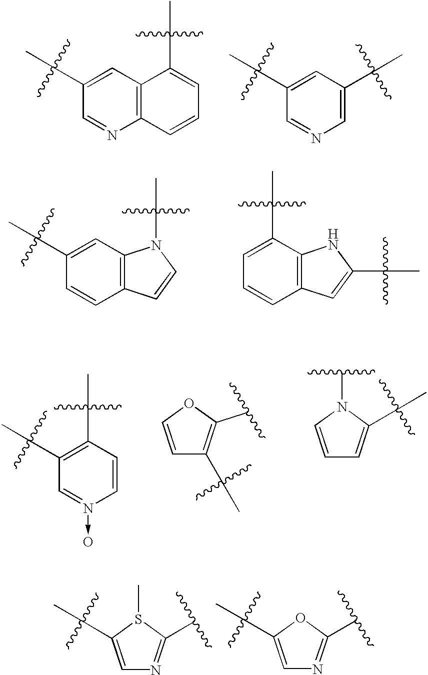

The term “heterocycloalkyl”, “heterocycloalkylene” as used herein represents a cycloalkyl, as defined above, wherein at least one and a maximum of six (6) carbon atoms is replaced by heteroatom selected from N, O and S. The heteroatoms can exist in their respective oxidized states. Thus a sulfur (S) atom can exist as a sulfoxide or sulfone, while a nitrogen (N) can exist as in the form of an N-oxide group. Illustrative examples of a heterocycloalkyl and heterocycloalkylene group or moiety are morpholine, imidazolinyl, dioxalanyl, piperidyl, pyrrolidinyl, pyrrolinyl, imidazolidinyl, quinuclidinyl, morpholinyl, [1,4′]bipiperidinylyl, pyrazolinyl, pyranyl, 1′,2′-dihydro-2H-[1,4′]bipyridinylyl, 1-morpholin-4-ylpiperidinyl, and the like. A heterocycloalkyl or heterocycloalkylene is substituted with one to four groups selected from hydrogen, halogen, nitro, cyano, C₁₋₈ straight chain alkyl, C₃₋₁₄ branched alkyl, —OR¹¹, —C(O)R¹¹, —C(O)OR¹¹, —C(O)NR¹¹R¹¹, —S(O)₂NR¹¹R¹¹, —X⁷NR¹¹R¹¹, —X⁷NR¹¹C(O)OR, —X⁷NR¹¹C(O)NR¹¹R¹¹ or —X⁷NR¹¹C(NR¹¹NR¹¹R¹¹, wherein X⁷ represents is a bond or —(CH₂)₁₋₆— and each R¹¹ independently is hydrogen or —(C₁₋₆)alkyl.

The following representations further illustrate the terms “heterocycloalkyl” and “heterocycloalkylene”.

Heterocycloalkyl:

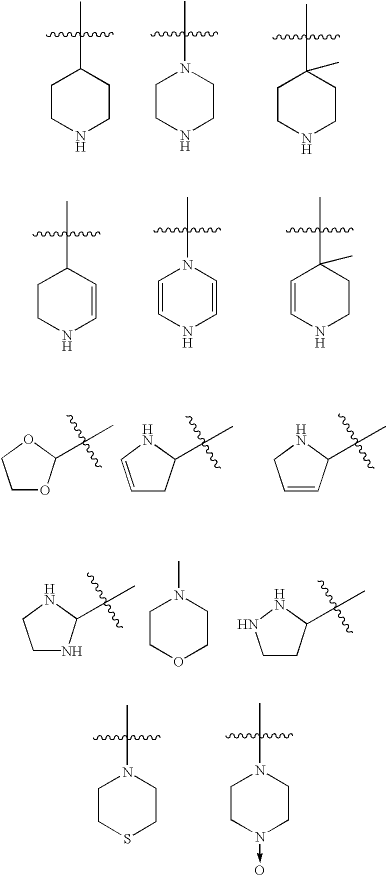

Heterocycloalkylene:

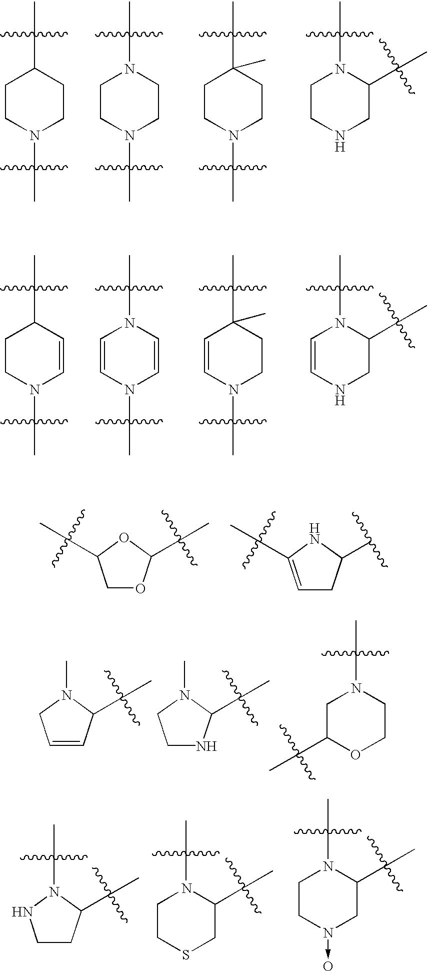

As used in the present invention, the illustration:

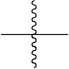

generally indicates a point of attachment of the group, comprising the illustration, to another group or atom.

The present invention also includes the prodrugs of compounds of Formula I. The term prodrug is intended to represent covalently bonded carriers, which are capable of releasing the active ingredient of Formula I when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I include compounds wherein a hydroxy, amidino, guanidino, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula I), amides (e.g, trifluoroacetylamino, acetylamino, and the like), and the like. Prodrugs of compounds of Formula I are also within the scope of this invention.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

The term “pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of Formula I which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, madelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolaline, triethanolamine, trimethamine, N-methylglucamine and the like.

The term “therapeutically effective amount” means that amount which, when administered to an animal for treating a disease, is sufficient to effect such treatment for the disease.

The terms “treatment” or “treating” means any administration of a compound of the present invention and includes:

preventing the disease from occurring in an animal which may be predisposed to the disease but does not yet experience or display the pathology or symptomatology of the disease,

inhibiting the disease in an animal that is experiencing or displaying the pathology or symptomatology of the diseased (i.e., arresting further development of the pathology and/or symptomatology), or

ameliorating the disease in an animal that is experiencing or displaying the pathology or symptomatology of the diseased (i.e., reversing the pathology and/or symptomatology).

Nomenclature:

The compounds of Formula I and the intermediates and starting materials used in their preparation are named in accordance with IUPAC rules of nomenclature in which the characteristic groups have decreasing priority for citation as the principle group as follows: acids, esters, amides, etc. or by using the “Autonom”, a Beilstein Commander 2.1 Application, distributed by Beilstein.

### Presently Preferred Embodiments

While the broadest definition of this Invention is set forth in the Summary of the Invention, certain aspects of the Invention are preferred.

Preferred embodiments of the present invention provide compounds of Formula I, wherein:

X¹ represents —(CH₂)₀₋₂—L—(CH₂)₀₋₂—Z²—X²;

Z represents arylene, heteroarylene or heterocycloalkylene;

X² represents H or —(CH₂)₀₋₂—L—(CH₂)₀₋₂—Z³—X³;

Z³ represents arylene, heteroarylene or heterocycloalkylene; and

X³ represents H or —(CH₂)₀₋₂—L—(CH₂)₀₋₂—Z⁴.

Another preferred embodiment provides compound wherein, Z¹ represents arylene or heteroarylene; X¹ represents —Z²—X² or —(CH₂)₀₋₁—O—(CH₂)₀₋₁—Z²—X^{2;} and L represents a bond, —O—, —SO₂—, —COO—, or —NR³⁵—.

A further preferred embodiment provides a compound wherein R⁴ represents C₁₋₈ alkyl, —(CH₂)₁₋₆-aryl, or —(CH₂)₁₋₆—S(O)ₙ—C₁₋₄-alkyl; R⁵ and R⁶ along with the carbon atom to which they are attached represent a —CO—group; Z¹ represents arylene; L represents a bond; and X¹ represents —Z²—X².

In yet another preferred embodiment are provided compounds wherein, R² represents H; R³ represents C₃₋₅ branched or straight chain unsubstituted alkyl; or R² and R³ along with the carbon atom to which they are attached form a five to six membered cycloalkyl or a five to six heterocycloalkyl moiety; R⁴ represents —C₁₋₅ alkyl, —(CH₂)₁₋₄-aryl, or —(CH₂)₁₋₄—S(O)ₙ—C₁₋₄-alkyl; and R⁷ represents a seven to nine membered bicyclic heteroaryl or a seven to nine membered bicyclic heterocycloalkyl moiety, said moieties substituted with R⁸, R⁹ and R¹⁰.

A further preferred embodiment provides compounds wherein, R³ represents i-butyl; R² and R³ along with the carbon atom to which they are attached form a six membered cycloalkyl moiety; R⁴ represents —C₂₋₄ alkyl, —(CH₂)₁₋₂-aryl, or —(CH₂)₁₋₂—S(O)ₙ—C₁₋₂-alkyl; and R⁷ represents a nine membered bicyclic heteroaryl or a nine membered bicyclic heterocycloalkyl moiety, said moieties substituted with R⁸, R⁹ and R¹⁰.

Particularly preferred groups are wherein, R³ represents C₃₋₅ branched or straight chain unsubstituted alkyl; R⁴ represents unsubstituted —C₂₋₄ alkyl, preferably n-propyl, —(CH₂)₁₋₂-phenyl, or —(CH₂)₂—S(O)ₙ—CH₃; R⁵ and R⁶ along with the carbon atom to which they are attached represent a —CO—group; Z¹ represents phenylene; X¹ represents —Z²—X² where Z² is phenylene and X² is —Z³—X³ where Z³ is heteroarylene or heterocycloalkylene and X³ is H or Z⁴; R⁷ represents an indolyl, benzoxazolyl or a benzimidazolyl moiety; and R^{8,} R⁹ and R¹⁰ independently represent a H or C₁₋₄ alkyl. Within this group, Q¹ and Q² are preferably a bond.

Particularly preferred compounds of the present invention are selected from:

4-Methyl-2-(4′-piperazin-1-yl-biphenyl-3-yl)-pentanoic acid [1-(benzooxazole-2-carbonyl)-butyl]-amide;

4-Methyl-2-(4′-piperazin-1-yl-biphenyl-4-yl)-pentanoic acid [1-(benzooxazole-2-carbonyl)-butyl]-amide;

4-Methyl-2-[4′-(1H-pyrrol-2-yl)-biphenyl-4-yl]-pentanoic acid [1-(benzooxazole-2-carbonyl)-butyl]-amide;

4-Methyl-2-[4′-(5-morpholin-4-yl-1H-pyrrol-2-yl)-biphenyl-4-yl]-pentanoic acid [1-(benzooxazole-2-carbonyl)-butyl]-amide;

4-Methyl-2-[4′-(5-piperazin-1-yl-1H-pyrrol-2-yl)-biphenyl-4-yl]-pentanoic acid [1-(benzooxazole-2-carbonyl)-butyl]-amide;

4-Methyl-2-[4′-(1-piperazin-1-yl-piperidin-4-yl)-biphenyl-4-yl]-pentanoic acid [1-(benzooxazole-2-carbonyl)-butyl]-amide;

4-Methyl-2-[4′-(4-piperidin-4-yl-piperazin-1-yl)-biphenyl-4-yl]-pentanoic acid [1-(benzooxazole-2-carbonyl)-butyl]-amide; and

4-Methyl-2-[4′-(4-piperidin-4-yl-piperazin-1-yl)-biphenyl-3-yl]-pentanoic acid [1-(benzooxazole-2-carbonyl)-butyl]-amide.

In yet another embodiment are provided compounds wherein, R³ represents H; and Q² is —NR¹¹— where R¹¹ and R² along with the nitrogen and carbon atoms to which R¹¹ and R² are respectively attached can be taken together to form a 5-6 membered heterocycloalkylene moiety optionally containing one to two heteroatoms selected from oxygen and nitrogen, preferably R¹¹ and R² along with the nitrogen and carbon atoms to which R¹¹ and R² are respectively attached form piperidine or piperazine.

Also provided in this embodiment are compounds wherein, R⁴represents C₁₋₅ alkyl; and R⁷ represents a nine membered bicyclic heteroaryl or a nine membered bicyclic heterocycloalkyl moiety, said moieties substituted with R⁸, R⁹ and R¹⁰.

Further preferred compounds are those wherein, R⁴ represents C₁₋₅ alkyl, —(CH₂)₁₋₄-aryl, or —(CH₂)₁₋₄—S(O)ₙ—C₁₋₄-alkyl; R⁷ represents a seven to nine membered bicyclic heteroaryl or a seven to nine membered bicyclic heterocycloalkyl moiety, said moieties substituted with R⁸, R⁹ and R¹⁰; R¹⁴ represents H; and Q² is NR¹¹ where R¹¹ and R² along with the nitrogen and carbon atoms to which R¹¹ and R² are respectively attached can be taken together to form a 5-6 membered heterocycloalkylene moiety containing one to two additional heteroatoms selected from oxygen and nitrogen, preferably R¹¹ and R² along with the nitrogen and carbon atoms to which R¹¹ and R² are respectively attached form piperidine or piperazine. Within this group a more preferred group of compounds is that wherein R⁴ represents unsubstituted —C₂₋₄ alkyl, preferably n-propyl, —(CH₂)₁₋₂-phenyl, or —(CH₂)₂—S(O)ₙ—CH₃; R⁵ and R⁶ along with the carbon atom to which they are attached represent a —CO—group; Z¹ represents phenylene or heteroarylene; X¹ represents —Z²—X² where z² is phenylene, heteroarylene, or heterocycloalkylene and X² is —Z³—X³ where Z is arylene, heteroarylene, or heterocycloalkylene and X³ is H or Z^{4;} R⁷ represents an indolyl, benzoxazolyl or a benzimidazolyl moiety; and R⁸, R⁹ and R¹⁰ independently represent a H or C₁₋₄ alkyl. Within this group, Q¹ is preferably a bond.

Particularly preferred compounds of this embodiment are selected from:

1-[4-(4-Piperazin-1-yl-phenyl)-thiazol-2-yl]-piperidine-2-carboxylic acid [1-(benzooxazole-2-carbonyl)-butyl]-amide;

1-(4′-Piperazin-1-yl-biphenyl-3-yl)-piperidine-2-carboxylic acid [1-(benzooxazole-2-carbonyl)-butyl]-amide;

1-(4′-Pyridin-3-yl-biphenyl-4-yl)-piperidine-2-carboxylic acid [1-(benzooxazole-2-carbonyl)-butyl]-amide;

1-(4-{2-[4-(4-Methyl-piperazin-1-yl)-phenyl]-thiazol-4-yl }-phenyl)-piperidine-2-carboxylic acid [1-(benzooxazole-2-carbonyl)-butyl]-amide;

1-{4-[2-(4-Methyl-piperazin-1-yl)-thiazol-4-yl]-phenyl}-piperidine-2-carboxylic acid [1-(benzooxazole-2-carbonyl)-butyl]-amide;

1-{2-[4-(4-Methyl-piperazin-1-yl)-phenyl]-thiazol-4-yl}-piperidine-2-carboxylic acid [1-(benzooxazole-2-carbonyl)-butyl]-amide;

6′-(4-Pyridin-3-yl-piperazin-1-yl)-3,4,5,6-tetrahydro-2H-[1,3′]bipyridinyl-2-carboxylic acid [1-(benzooxazole-2-carbonyl)-butyl]-amide;

1-(4′-Pyridin-3-yl-biphenyl-4-yl)-piperidine-2-carboxylic acid [1(S)-(benzooxazole-2-carbonyl)-butyl]-amide;

1-{4-[4-(4-Methyl-[1,4]diazepan-1-ylamino)-piperidin-1-yl]-phenyl }-piperidine-2-carboxylic acid [1-(benzooxazole-2-carbonyl)-butyl]-amide; and

1-{4-[6-(1-Methyl-piperidin-4-ylamino)-pyridin-3-yl]-thiophen-3-yl }-piperidine-2-carboxylic acid [1-(benzooxazole-2-carbonyl)-butyl]-amide.

### EXAMPLES

The following preparations and examples are given to enable those skilled in the art to more clearly understand and to practice the present invention. They should not be considered as limiting the scope of the invention, but merely as being illustrative and representative thereof.

General Synthetic Procedures:

Compounds of the present invention can be prepared by the procedures outlined in synthetic Scheme I below.

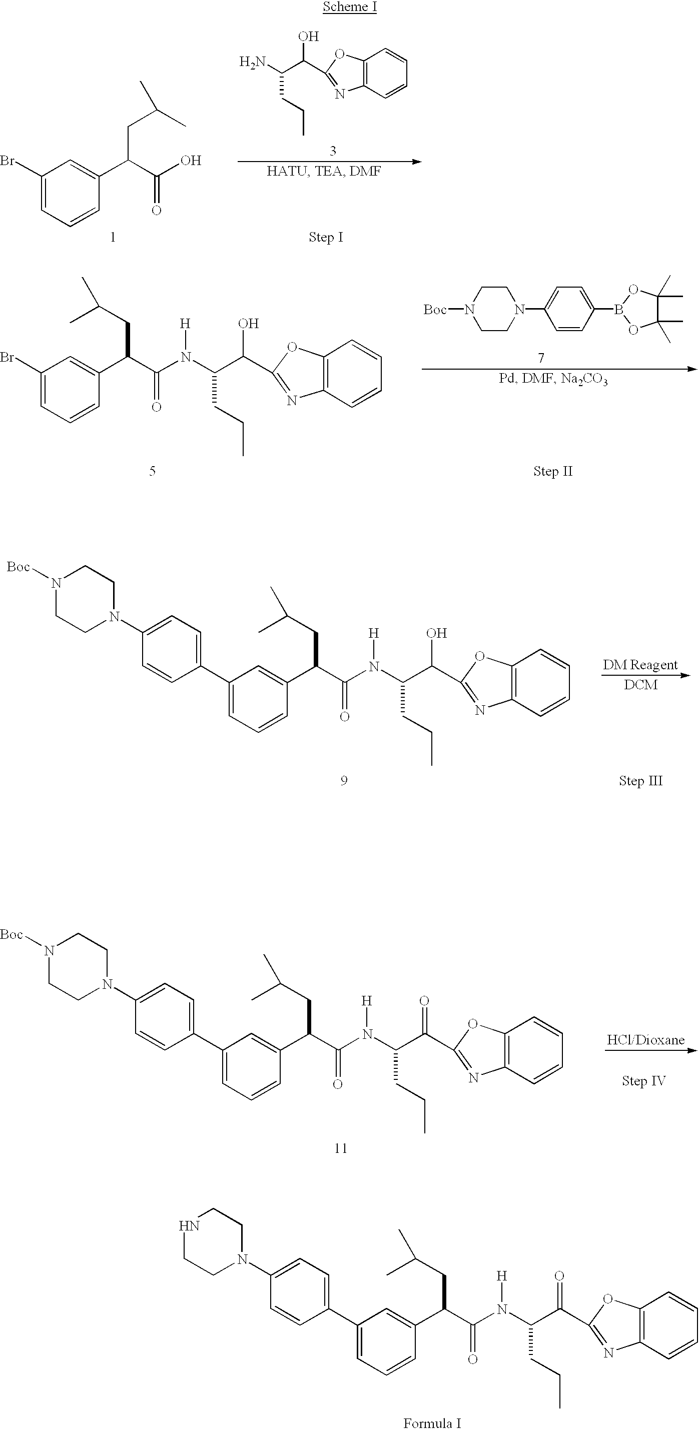

Step I

A mixture of a compound of the acid of formula 1 (540 mgs, 2.0 mmol) and dry DMF (5 mL) was agitated at ambient temperature in a nitrogen atmosphere. This mixture then was combined with an amine (HATU, 440 mg, 2.0 mmol) and the resulting reaction mixture was cooled to about 0° C. The cooled reaction mixture then was mixed with PyBop PyBOP (1.0 gm, 2.0 mmol) and triethylamine (400 μl, 3.0 mmol) and the then resulting reaction mixture was agitated at ambient temperature feom about 12 to about 18 hours. The agitated reaction mixture then was mixed with saturated aqueous sodium carbonate (10 mL) and ethyl acetate (50 mL). The organic layer was isolated and sequentially washed with water (x1), brine (x1), dried (anhydrous magnesium sulfate) and concentrated under reduced pressure to yield a residue. The residue was purified by flash column on silica gel with 50% hexane in ethyl acetate as eluent to yield a compound of formula 5 in about a 50% yield. ¹H NMR (dmso-d6, ppm) δ: 0.85 (t, 3 H0, 1.01 (d, 6 H), 1.33-1.81 (m, 7 H), 3.31 (m, 1 H), 3.71 (m, 1 H), 4.11 (m, 1 H), 5.12 (m, 1 H), 7.06-7.36 (m, 7 H).

Step II

The compound of formula 5 (340 mgs, 0.75 mmol) and the compound of formula 7 (340 mgs, 1.0 mmol) were combined with a mixture of DMF (2 mL) and saturated quueous sodium bicarbonate (1 mL). The resulting reaction mixture was purged with nitrogen gas for about 2 minutes and then Pd (dppf)₂CH₃CN (50 mg.) was added and the resulting mixture was agitated at a temperarture of about 90° C. for about 2 hours. The agitated reaction mixture was cooled to ambient temperature and diluted with ethyl acetate (50 mL). The organic layer was isolated, washed with water, dried (MgSO₄) and concentrated under reduced pressure to yield a residue. The residue was purified by column chromatography using silica gel with 50% hexane in ethyl acetate as an eluent to yield the compound of formula 9 in about a 10% yield. Fab LC-MS 655 (M+H⁺).

Step III

The compound of formula 9 (300 mgs, 0.48 mmol) was dissolved in dry dichloromethane (DCM, 2 mL) to form a mixture. The mixture was cooled to about 0° C. and the cooled mixture was diluted with Dess-martin reagent (DM Reagent, 300 mgs, 1.2 mmol) with stirring to form a reaction mixture. The reaction mixture turned colors (to an orange color) in about 15 minutes. The colored reaction mixture was diluted with isopropanol (0.5 ml) and the resulting mixture was agitated for about 10 minutes. The agitated reaction mixture was diluted with saturated aqueous sdoium bicarbonate (5 mL) and ethyl acetate. The oragnic layer was isolated, washed with water, dried (MgSO₄), passed through a plug of silica gel, and concentrated under reduced pressure to yield the compound of formula 11 in about a 66% yield. ¹H NMR (dmso-d6, ppm) δ: 0.86 (t, 3 H), 0.92 (d, 6 H), 1.33-1.86 (m, 7 H), 1.42 (s, 9 H), 2.77 (m, 4 H), 3.47 (m, 4 H), 4.11 (m, 1 H), 4.5 (m, 1 H), 6.71 (m, 2 H), 7.11 (m, 1 H), 7.27-7.38 (m, 8 H).

Step IV

4-Methyl-2-(4′-piperazin-1-yl-biphenyl-3-yl)-pentanoic acid [1-(benzooxazole-2-carbonyl)-butyl]-amide

The compound of formula 11 (200 mgs, 0.33 mmol) and 4.0 Molar HC1 in dioxane (about 0.5 mL) were mixed to form a mixture. The mixture was agitated for about 10 minutes. The agitated reaction mixture then was diluted with ether (20 mL) leading to the formation of a solid. The solid was isolated and air dried to yield the compound of Formula I in about a 100% yield. ¹H NMR (dmso-d6, ppm) δ: 0.86 (t, 3 H), 0.92 (d, 6 H), 1.33-1.86 (m, 7 H), 3.11 (m, 4 H), 3.47 (m, 4 H), 4.15 (m, 1 H), 4.45 (m, 1 H), 6.71 (m, 2 H), 7.11 (m, 1 H), 7.27-7.38 (m, 8 H). FAB LC-MS 553.1 (m+H⁺).

The following compounds of Formula I can be prepared by the process discussed above or by methods known to one skilled in the art.

4-Methyl-2-(3-pyridin-3-yl-phenyl)-pentanoic acid [1-(benzooxazole-2-carbonyl)-butyl]-amide. ¹H NMR (DMSO-d_{6,} ppm) δ: 0.81 (m, 9 H), 1.31-1.81 (m, 7 H), 3.80 (m, 1 H), 5.12 (m, 1 H), 7.47-7.99 (m, 11 H), 8.55 (m, 1 H), 8.77 (m, 2 H). LC-MS (FAB) 470.2 (M+H⁺).

4-Methyl-2-{4′-[2-(4-methyl-piperazin-1-yl)-thiazol-4-yl]-biphenyl-3-yl }-pentanoic acid [1-(benzooxazole-2-carbonyl)-butyl]-amide. ¹H NMR (DMSO-d_{6,} ppm) δ: 0.88 (m, 9 H), 1.34.-1.81 (m, 7 H), 2.86 (s, 3 H), 3.44-3.61 (m, 4 H), 3.88 (m, 4 H), 3.91 (m, 1 H), 4.45 (m, 1 H), 7.46 (m, 12, H), 8.81 (m, 1 H). LCMS (FAB) 650.1 (M+H⁺).

1-(4′-Piperazin-1-yl-biphenyl-3-ylamino)-cyclohexanecarboxylic acid

[1-(benzooxazole-2-carbonyl)-butyl]-amide. ¹HNMR (DMSO-d₆) δ: 9.08 (1H, m), 8.22 (1H, d), 7.90 (1H, d), 7.81 (1H, d), 7.57 (1H, m), 7.47 (1H, m), 7.35 (2H, d), 6.93 3H, m), 6.76 (2H, m), 6.48 (1H, d), 5.17 (1H, m), 3.33 (4H, m), 3.16 (4H, m), 1.39 (14H, m), 0.66 (3H, t).

LC/MS, M+1:580.6.

4-Methyl-2-(4′-piperazin-1-yl-biphenyl-4-ylsulfanyl)-pentanoic acid [1-(benzooxazole-2-carbonyl)-butyl]-amide. ¹HNMR (DMSO-d₆) δ: 8.60 (1H, d), 7.93 (2H, d), 7.80 (2H, m), 7.47 (8H, m), 7.00 (2H, m), 5.10 (1H, m), 3.88 (1H, m), 3.40 (4H, m), 3.15 (4H, m), 1.40 (7H, m), 0.75 (9H, m). LC/MS, M+1:585.4.

Additional Processes for Preparing Compounds of Formula I:

A compound of Formula I can be prepared as a pharmaceutically acceptable acid addition salt by reacting the free base form of the compound with a pharmaceutically acceptable inorganic or organic acid. Alternatively, a pharmaceutically acceptable base addition salt of a compound of Formula I can be prepared by reacting the free acid form of the compound with a pharmaceutically acceptable inorganic or organic base. Inorganic and organic acids and bases suitable for the preparation of the pharmaceutically acceptable salts of compounds of Formula I are set forth in the definitions section of this application. Alternatively, the salt forms of the compounds of Formula I can be prepared using salts of the starting materials or intermediates.

The free acid or free base forms of the compounds of Formula I can be prepared from the corresponding base addition salt or acid addition salt form. For example, a compound of Formula I in an acid addition salt form may be converted to the corresponding free base by treating with a suitable base (e.g., ammonium hydroxide solution, sodium hydroxide, or the like). A compound of Formula I in a base addition salt form can be converted to the corresponding free acid by treating with a suitable acid (e.g., hydrochloric acid, or the like).

The N-oxides of compounds of Formula I can be prepared by methods known to those of ordinary skill in the art. For example, N-oxides can be prepared by treating an unoxidized form of the compound of Formula I with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as methylene chloride) at approximately 0° C. Alternatively, the N-oxides of the compounds of Formula I can be prepared from the N-oxide of an appropriate starting material.

Compounds of Formula I in unoxidized form can be prepared from N-oxides of compounds of Formula I by treating with a reducing agent (e.g. sulfur, sulfur dioxide, triphenyl phosphine, lithium borohydride, sodium borohydride, phosphorus trichloride, tribromide, or the like) in a suitable organic solvent (e.g., acetonitrile, ethanol, aqueous dioxane, or the like) at 0 to 80° C.

Prodrug derivatives of the compounds of Formula I can be made by means known to those of ordinary skill in the art (e.g., for further details see Saulnier et al. (1994), Bioorganic and Medicinal Chemistry Letters. 4: 1985). For example, appropriate drugs can be prepared by reacting a non-derivatized compound of Formula I with a suitable carbamylating agent (e.g., 1,1-acyloxyalkylcarbonochloridate, para-nitrophenyl carbonate, or the like).

Protected derivatives of the compounds of Formula I can be made by means known to those of ordinary skill in the art. A detailed description of the techniques applicable to the creation of protective groups and their removal can be found in T. W. Greene, Protective Groups in Organic Synthesis, John Wiley & Sons, Inc. 1981.

Compounds of Formula I can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds of Formula I, dissociable complexes are preferred (e.g., crystalline diastereoisomeric salts). Diastereomers have distinct physical properties (e.g., melting points, boiling points, solubilities, reactivity, and the like) and can be readily separated by taking advantage of these dissimilarities. The diastereomers can be separated by chromatography or, preferable, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

Pharmacology and Utility:

The compounds of this invention are cysteine protease inhibitors. In particular the compounds of this invention inhibit the activity of cathepsin K and, as such, are useful for treating diseases in which cathepsin K activity contributes to the pathology and/or symptomatology of the disease. For example, the compounds of this invention are useful in treating bone resorption disorders, e.g., osteoporosis. The compounds of this invention also are useful in treating autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis. The compounds of this invention also are useful in treating allergic disorders, including, but not limited to asthma; and allogeneic immune responses, including, but not limited to, organ transplants or tissue grafts. In particular, the compounds of this invention are useful in treating osteoporosis in humans by inhibition of cathepsin K, particularly in treating post-menopausal women.

The cysteine protease inhibitory activities of the compounds of the invention can be determined by methods known to those of ordinary skill in the art. Suitable in vitro assays for measuring protease activity and the inhibition thereof by test compounds are known. Typically, the assay measures protease induced hydrolysis of a peptide based substrate. Details of assays for measuring protease inhibitory activity are set forth below.

Administration and Pharmaceutical Compositions:

In general, compounds of Formula I will be administered in therapeutically effective amounts via any of the usual and acceptable modes known in the art. A therapeutically effective amount may vary widely depending on the severity of the disease, the age and relative health of the subject, the potency of the compound used and other factors. For example, therapeutically effective amounts of a compound of Formula I may range from 0.1 micrograms per kilogram body weight (μg/kg) per day to 10 milligram per kilogram body weight (mg/kg) per day, typically 1 μg/kg/day to 1 mg/kg/day. Therefore, a therapeutically effective amount for a 80 kg human patient may range from 10 μg/day to 100 mg/day, typically 0.1 mg/day to 10 mg/day. In general, one of ordinary skill in the art, acting in reliance upon personal knowledge and the disclosure of this Application, will be able to ascertain a therapeutically effective amount of a compound of Formula I for treating a given disease.

The compounds of Formula I can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula I in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, or the like). Preferred liquid carriers, particularly for injectable solutions, include water, saline, aqueous dextrose and glycols.

The amount of a compound of Formula I in the composition may vary widely depending upon the type of formulation, size of a unit dosage, kind of excipients and other factors known to those of skill in the art of pharmaceutical sciences. In general, a composition of a compound of Formula I for treating a given disease will comprise from 0.01% w to 10% w, preferably 0.3% w to 1% w, of active ingredient with the remainder being the excipient or excipients. Preferably the pharmaceutical compositions is administered in a single unit dosage form for continuous treatment or in a single unit dosage form ad libitum when relief of symptoms is specifically required. Representative pharmaceutical formulations containing a compound of Formula I are described below.

The compounds of Formula I can be administered alone or in combination with other compounds of Formula I or in combination with one or more other active ingredient(s). For example, the compounds of Formula I can be administered in combination with a therapeutically active amount of a bisphosphonic acid or acid ester derivative or any pharmaceutically acceptable salt thereof. Suitable bisphosphonic acids and acid ester derivatives include compounds corresponding to the following formula:

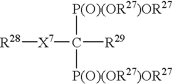

wherein X⁷ is a bond or (C₁₋₇)alkylene, each R²⁷ independently is hydrogen or (C₁₋₃₀)alkyl, R²⁸ and R²⁹ are selected independently from a group consisting of hydrogen, halo, optionally substituted (C₁₋₃₀)alkyl, (C₃₋₃₀)cycloalkyl, hetero(C₅₋₃₀)cycloalkyl, optionally substituted (C₆₋₁₀)aryl, hetero(C₆₋₁₀)aryl, NR³⁰R³⁰, OR³⁰, SR³⁰, wherein each R³⁰ independently is hydrogen, (C₁₋₁₀)alkyl, (C₃₋₁₀)cycloalkyl, optionally substituted (C₆₋₁₀)aryl, provided that both R²⁸ and R²⁹ are not selected from hydrogen or hydroxy when X⁷ is a bond; or R²⁸ and R²⁹ taken together form (C₂₋₉)alkylene; wherein (C₃₋₁₀)cycloalkyl includes adamantyl and the like, hetero(C₅₋₁₀)cycloalkyl includes pyrrolidinyl and the like, (C₆₋₁₀)aryl includes phenyl and naphthyl, and hetero(C₆₋₁₀)aryl includes quinolyl, isoquinolyl, pyridyl, furyl, imidazolyl, imidazopyridyl and the like.

Instances wherein R²⁸ and/or R²⁹ are substituted (C₁₋₃₀)alkyl may include, but are not limited to, (C₁₋₃₀)alkyl substituted by hetero(C₅₋₁₀)cycloalkyl, (C₆₋₁₀)aryl, hetero(C₆₋₁₀)aryl, NR³¹R³¹, OR³¹ and SR³¹, wherein each R³¹ is independently hydrogen or (C₁₋₁₀)alkyl; wherein hetero(C₅₋₁₀)cycloalkyl includes pyrrolidinyl and the like, (C₆₋₁₀)aryl includes phenyl and naphthyl, and hetero(C₆₋₁₀)aryl includes quinolyl, isoquinolyl, pyridyl, furyl, imidazolyl, imidazopyridyl and the like. Suitable optionally substituted aryl groups include, but are not limited to, halo-substituted phenyl.

A non-limiting class of bisphosphonic acids and acid ester derivatives thereof suitable for administration in combination with compounds of Formula I include those in which R²⁸ is selected from the group consisting of hydrogen, hydroxy or halo, and R²⁹ is selected from the group consisting of optionally substituted (C₁₋₃₀)alkyl, halo and SR³⁰, wherein R³⁰ is (C₁₋₁₀)alkyl or phenyl.

A non-limiting subclass of bisphosphonic acids and acid ester derivatives thereof suitable for administration in combination with compounds of Formula I include those in which R²⁸ is selected from the group consisting of hydrogen, hydroxy and chloro and R²⁹ is selected from the group consisting of optionally substituted (C₁₋₃₀)alkyl, chloro and chlorophenylthio.

A non-limiting example of a bisphosphonic acid suitable for administration in combination with compounds of Formula I include that in which X⁷ is a bond, each R²⁷ is hydrogen, R²⁸ is hydroxy and R²⁹ is 3-aminopropyl, namely 4-amino-1-hydroxybutylidene-1,1-bisphosphonic acid (aka alendronic acid), or the monosodium trihydrate salt thereof, namely 4-amino-1-hydroxybutylidene-1,1-bisphosphonate monosodium trihydrate (aka alendronate monosodium trihydrate), described in U.S. Pat. Nos. 4,922,007, to Kieczykowski et al., issued May 1, 1990; 5,019,651, to Kieczykowski et al., issued May 28, 1991; 5,510,517, to Dauer et al., issued Apr. 23, 1996; 5,648,491, to Dauer et al., issued Jul. 15, 1997, all of which patents are incorporated by reference herein in their entirety.

Further non-limiting examples of bisphosphonic acids suitable for administration in combination with compounds of Formula I include the following:

cycloheptylaminomethylene-1,1-bisphosphonic acid (aka cimadronic acid), described in U.S. Pat. No. 4,970,335, to Isomura et al., issued Nov. 13, 1990; 1,1-dichloromethylene-1,1-diphosphonic acid (aka clodronic acid) and the disodium salt thereof, namely clodronate disodium, described in Belgium Patent 672,205 (1966) and J. Org. Chem 32, 4111 (1967); 1-hydroxy-3-pyrrolidin-1-ylpropylidene-1,1-bisphosphonic acid (aka EB-1053); 1-hydroxyethylidene-1,1-diphosphonic acid (aka etidronic acid); 1-hydroxy-3-(N-methyl-N-pentylamino)propylidene-1,1-bisphosphonic acid (aka ibandronic acid), described in U.S. Pat. No. 4,927,814, issued May 22,1990; 6-amino-1-hydroxyhexylidene-1,1-bisphosphonic acid (aka neridronic acid); 3-(dimethylamino)-1-hydroxypropylidene-1,1-bisphosphonic acid (aka olpadronic acid); 3-amino-1-hydroxypropylidene-1,1-bisphosphonic acid (aka pamidronic acid); 2-pyrid-2-ylethylidene-1,1-bisphosphonic acid (aka piridronic acid), described in U.S. Pat. No. 4,761,406; 1-hydroxy-2-pyrid-3-ylethylidene-1,1-bisphosphonic acid (aka risedronic acid); 4-chlorophenylthiomethylenebisphosphonic acid (aka tiludronic acid), described in U.S. Pat. No. 4,876,248, to Breliere et al., Oct. 24, 1989; and 1-hydroxy-2-(1H-imidazol-1-yl)ethylidene-1, 1-bisphosphonic acid (aka zoledronic acid); all of which patents and other documents referred to above are incorporated by reference herein in their entirety.

A non-limiting subclass of bisphosphonic acids suitable for administration in combination with compounds of Formula I include those selected from the group consisting of alendronic acid, cimadronic acid, clodronic acid, tiludronic acid, etidronic acid, ibandronic acid, risedronic acid, piridronic acid, pamidronic acid, zolendronic acid, pharmaceutically acceptable salts thereof, and mixtures thereof. A further example of a bisphosphonic acid suitable for administration in combination with compounds of Formula I is alendronic acid or a pharmaceutically acceptable salt thereof, and mixtures thereof. A further non-limiting example is alendronate monosodium trihydrate.

Compounds of Formula I can be administered in combination with a therapeutically active amount of an estrogen receptor agonist. Non-limiting examples of estrogen receptor agonists suitable for administration in combination with the compounds of Formula I include naturally occurring estrogens such as estradiol, estrone and estroil, or synthetic estrogen receptor agonists such as [6-hydroxy-2-(4-hydroxyphenyl)benzo[b]thien-3-yl][4-(2-piperidin-1-ylethoxy)phenyl]methanone (aka raloxifene) and {2-[4-(1,2-diphenylbut-1-enyl)-phenoxy]ethyl}dimethylamine (aka tamoxifen). A non-limiting subclass of estrogen receptor agonists suitable for administration in combination with the compounds of Formula I include estrogen receptor partial agonists (i.e., estrogen receptor agonists with mixed agonist/antagonist properties), sometimes referred to as estrogen receptor modulators. Estrogen receptor partial agonists can exert tissue-selective estrogen agonist effects. Tamoxifen, for example, selectively * exerts an estrogen agonist effect on the bone, in humans. Additional suitable estrogen receptor partial agonists are described in Tissue-Selective Actions Of Estrogen Analogs, Bone Vol. 17, No. 4, Oct. 1995, 181S-190S. Certain 3-[4-(2-phenylindol-1-ylmethyl)phenyl]acrylamides, described in U.S. Pat. No. 5,985,910 to Miller et al., Nov. 16, 1999; benzothiphene compounds, described in U.S. Pat. No. 5,985,897 to Meuhl et al., Nov. 16, 1999; naphthyl compounds, described in U.S. Pat. No. 5,952,350 to Cullinan et al., Sep. 14, 1999; substituted benzothiophene compounds, described in U.S. Pat. No. 5,962,475 to Schmid et al., Oct. 4, 1999, are suitable estrogen receptor partial agonists for administration with the compounds of Formula I; all of which patents and other documents referred to above are incorporated by reference herein in their entirety.

More particularly a pharmaceutical composition of this invention may comprise a therapeutically effect amount of a compound of Formula I in combination with one or more active ingredient(s) selected from the group consisting of (i) a therapeutically effect amount of a bisphosphonic acid or acid ester thereof or a pharmaceutically acceptable salt thereof and (ii) a therapeutically effect amount of an estrogen receptor agonist or a pharmaceutically acceptable salt thereof; and one or more pharmaceutically acceptable excipient(s). Non-limiting examples of such bisphosphonic acids include 1,1-dichloromethylene-1,1-diphosphonic acid, 1-hydroxy-3-pyrrolidin-1-ylpropylidene-1,1-bisphosphonic acid, 1-hydroxyethylidene-1,1-diphosphonic acid, 1-hydroxy-3-(N-methyl-N-pentylamino)propylidene-1,1-bisphosphonic acid, 6-amino-1-hydroxyhexylidene-1,1-bisphosphonic acid, 3-(dimethylamino)-1-hydroxypropylidene-1,1-bisphosphonic acid, 3-amino-1-hydroxypropylidene-1,1-bisphosphonic acid, 2-pyrid-2-ylethylidene-1,1-bisphosphonic acid, 1-hydroxy-2-pyrid-3-ylethylidene-1,1-bisphosphonic acid, 4-chlorophenylthiomethylenebisphosphonic acid and 1-hydroxy-2-(1H-imidazol-1-yl)ethylidene-1,1-bisphosphonic acid or acid ester thereof or a pharmaceutically acceptable salt thereof; particularly 1,1-dichloromethylene-1,1-diphosphonic acid or a pharmaceutically acceptable salt thereof and preferably 1,1-dichloromethylene-1,1-diphosphonate monosodium trihydrate.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound of Formula I 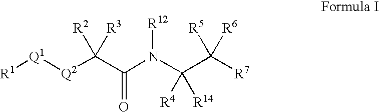
wherein:
R¹ represents —(CH₂)₀₋₂—Z¹—X¹;
Q¹—(CH₂)₀₋₁;
Q² represents a bond, S(O)₂, O or NR¹¹;
R² represents H;
R³ represents H or —C₃₋₆ alkyl; alternatively
R² and R³ along with the carbon atom to which they are attached form a three to eight membered cycloalkylene or heterocycloalkylene moiety;
R⁴ represents —C₁₋₈ alkyl, —(CH₂)₁₋₆-aryl, —(CH₂)₁₋₆—S(O)ₙ—C₁₋-alkyl, —(CH₂)₁₋₆-cycloalkyl, —(CH₂)₁₋₆—S(O)ₙ-cycloalkyl, —(CH₂)₁₋₆—S(O)ₙ—C₁₋₄-aryl, —(CH₂)₁₋₆-heteroaryl, —(CH₂)₁₋₆-S(O)ₙ—C₁₋₄-heteroaryl, —(CH₂)₁₋₆-heterocycloalkyl, —(CH₂)₁₋₆—NR^{a}R^{b} (where R^{a} and R^{b} are independently hydrogen, —C₁₋₆ alkyl, or C(═NH)—NH₂), or —(CH₂)₁₋₆—S(O)ₙ—C₁₋₄-heterocycloalkyl;
R¹⁴ represents H; alternatively
R⁴ and R¹⁴ along with the carbon atom to which they are attached form a three to eight membered cycloalkylene or heterocycloalkylene moiety;
R⁵ represents H;
R⁶ represents —OH; alternatively
R⁵ and R⁶ along with the carbon atom to which they are attached represent a —CO—group;
R⁷ represents a five to ten membered heteroaryl or a five to ten membered heterocycloalkyl moiety, said moieties substituted with R⁸, R⁹, and R¹⁰;
R¹¹ represents H, —C₁₋₆-alkyl, —C₁₋₆ alkyl substituted with hydroxy, alkoxy, or 2-hydroxyethyl; alternatively
R¹¹ and R² along with the nitrogen and carbon atoms to R¹¹ and R² are respectively attached can be taken together to form a 5-6 membered heterocycloalkylene moiety optionally containing one to three additional heteroatoms selected from nitrogen, oxygen and sulfur;
R¹² represents H or —C₁₋₆-alkyl;
Z¹ represents arylene, heteroarylene, cycloalkylene or heterocycloalkylene;
X¹ represents H or —(CH₂)₀₋₃—L—(CH₂)₀₋₃—Z²—X²;
n represents an integer from zero to two; and
R^{8,} R⁹ and R¹⁰ independently represent H, alkyl, cycloalkyl, halogen, nitro, cyano, —OH, —OR^{17,}—S(O)ₙ—-alkyl, —NH-alkyl, —N(alkyl)₂, —COOR¹⁷, —COOH, —SO₂N(R¹⁸)₂, —SO₂NR¹⁷R¹⁸, —CON(R¹⁸)₂, —CONR¹⁷R¹⁸, —CONR¹⁸COOH, —CONR¹⁸COOR¹⁷, aryl, a five to ten membered heteroaryl or a five to ten membered heterocycloalkyl moiety;
Z² represents arylene, heteroarylene, cycloalkylene or heterocycloalkylene;
X² represents H or —(CH₂)₀₋₃—L—(CH₂)₀₋₃—Z³—X³;
R¹⁷ represents H or —(C₁₋₆)alkyl, wherein said —(C₁₋₆)alkyl is substituted with zero to four substituents selected from halogen, —OR³², —SR³², —S(O)R³², —S(O)₂R³², —C(O)R³², C(O)OR³², —NR³²R³³, —NR³³C(O)OR³², —C(O)NR³²R³³, —S(O)₂NR³²R³³, —NR³³C(O)NR³²R³³, —NR³³C(NR³³ )NR³²R³³, —(CH₂)₀₋₆-cycloalkyl, —(CH₂)₀₋₆-diphenyl, —(CH₂)₀₋₆-heterocycloalkyl, —(CH₂)₀₋₆-aryl, —(CH₂)₀₋₆-heteroaryl or —(CH₂)₀₋₆-diheteroaryl;
R¹⁸ represents aryl, H, —C₁₋₆ alkyl, cycloalkyl or heteroaryl;
Z³ represents arylene, heteroarylene, cycloalkylene, or heterocycloalkylene;
X³ represents H or —(CH₂)₀₋₃—L—(CH₂)₀₋₃—Z⁴;
L represents a bond, —S(O)ₙ—, —COO—, —O—CO—, —NR³⁵—SO₂—, —CO—NR³⁵—SO₂NR³⁵—, —NR³⁵—CO—, —CO—, —NR³⁵—COO—, —COONR³⁵—, —NR³⁵—, —O—, or NR³⁵—C(NR³⁵)—;
R³² represents hydrogen, —C₁₋₆-alkyl, —(CH₂)₀₋₃-cycloalkyl, —(CH₂)₀₋₆-heterocycloalkyl, —(CH₂)₀₋₆-aryl, (CH₂)₀₋₆-diphenyl, —(CH₂)₀₋₆-heteroaryl, or —(CH₂)₀₋₆-diheteroaryl;
each R³³ represents hydrogen, —C₁₋₆-alkyl, —(CH₂)₀₋₃-cycloalkyl, —(CH₂)₀₋₆-heterocycloalkyl, —(CH₂)₀₋₆-aryl, or —(CH₂)₀₋₆-heteroaryl;
each R³⁵ represents H or C₁₋₄ alkyl; and
Z⁴ represents aryl, heteroaryl, cycloalkyl, or heterocycloalkyl; or a pharmaceutically acceptable salt thereof.

2. The compound of claim 1 wherein:
X¹ represents (CH₂)₀₋₂—L—(CH₂)₀₋₂—Z²—X²;
Z² represents arylene, heteroarylene or heterocycloalkylene;
X² represents H or (CH₂)₀₋₂—L—(CH₂)₀₋₂—Z³—X³;
Z³ represents arylene, heteroarylene or heterocycloalkylene; and
X³ represents H or (CH₂)₀₋₂—-L—(CH₂)₀₋₂—Z⁴.

3. The compound of claim 2 wherein:
Z¹ represents arylene or heteroarylene;
X¹ represents Z²—X² or (CH₂)₀₋₁—O—(CH₂)₀₋₁—, —Z²—X²; and
L represents a bond, —O—, —SO₂—, —COO—, or —NR³⁵—.

4. The compound of claim 3 wherein:
R⁴ represents C₁₋₈ alkyl, (CH₂)₁₋₆-aryl, or (CH₂)₁₋₆—S(O)ₙ—C₁₋₄-alkyl;
R⁵ and R⁶ along with the carbon atom to which they are attached represent a —CO—group;
L represents a bond; and
X¹ represents Z²—X².

5. The compound of claim 4 wherein:
R² represents H;
R³ represents C₃₋₅ branched or straight chain alkyl; or
R² and R³ along with the carbon atom to which they are attached form a five to six membered cycloalkyl or a five to six heterocycloalkyl moiety;
R⁴ represents C₁₋₅ alkyl, (CH₂)₁₋₄-aryl, or (CH₂)₁₋₄—S(O)ₙ—C₁₋₄-alkyl; and
R⁷ represents a seven to nine membered bicyclic heteroaryl or a seven to nine membered bicyclic heterocycloalkyl moiety, said moieties substituted with R⁸, R⁹ and R¹⁰.

6. The compound of claim 5 wherein:
R³ represents i-butyl; or
R² and R³ along with the carbon atom to which they are attached form a six membered cycloalkyl moiety;
R⁴ represents C₂₋₄ alkyl, (CH₂)₁₋₂-aryl, (CH₂)₁₋₂—S(O)ₙ—C₁₋₂-alkyl; and
R⁷ represents a nine membered bicyclic heteroaryl or a nine membered bicyclic heterocycloalkyl moiety, said moieties substituted with R⁸, R⁹ and R¹⁰.

7. The compound of claim 6 wherein:
R⁴ represents C₂₋₄ alkyl, (CH₂)₁₋₂-phenyl, (CH₂)₂—S(O)_{n—CH}₃;
Z¹ represents phenylene;
R⁷ represents an indolyl, benzoxazolyl or a benzimidazolyl moiety; and
R⁸, R⁹ and R¹⁰ independently represent H or C₁₋₄ alkyl.

8. The compound of claim 1 selected from the group consisting of:
4-Methyl-2-(4′-piperazin-1-yl-biphenyl-3-yl)-pentanoic acid [1-(benzooxazole-2-carbonyl)-butyl]-amide;
4-Methyl-2-(4′-piperazin-1-yl-biphenyl-4-yl)-pentanoic acid [1-(benzooxazole-2-carbonyl)-butyl]-amide;
4-Methyl-2-[4′-(1H-pyrrol-2-yl)-biphenyl-4-yl] -pentanoic acid [1-(benzooxazole-2-carbonyl)-butyl]-amide;
4-Methyl-2-[4′-(5-morpholin-4-yl-1H-pyrrol-2-yl)-biphenyl-4-yl]-pentanoic acid [1-(benzooxazole-2-carbonyl)-butyl]-amide;
4-Methyl-2-[4′-(5-piperazin-1-yl-1H-pyrrol-2-yl)-biphenyl-4-yl]-pentanoic acid [1(benzooxazole-2-carbonyl)-butyl]-amide;
4-Methyl-2-[4′-(1-piperazin-1-yl-piperidin-4-yl)-biphenyl-4-yl]-pentanoic acid [1-(benzooxazole-2-carbonyl)-butyl]-amide;
4-Methyl-2-[4′-(4-piperidin-4-yl-piperazin-1-yl)-biphenyl-4-yl]-pentanoic acid [1-(benzooxazole-2-carbonyl)-butyl]-amide; and
4-Methyl-2-[4′-(4-piperidin-4-yl-piperazin-1-yl)-biphenyl-3-yl]-pentanoic acid [1-(benzooxazole-2-carbonyl)-butyl]-amide.

9. The compound of claim 4 wherein:
R³ represents H; and
Q² is NR¹¹ where R¹¹ and R² along with the nitrogen and carbon atoms to which R¹¹ and R² are respectively attached can be taken together to form a 5-6 membered heterocycloalkylene optionally containing one to two additional heteroatoms selected from oxygen and nitrogen.

10. The compound of claim 9 wherein: p1 R¹¹ and R² along with the nitrogen and carbon atoms to which R¹¹ and R² are respectively attached can be taken together to form a 6 membered heterocycloalkylene optionally containing one additional nitrogen atom.

11. The compound of claim 10 wherein:
R⁴ represents C₁₋₅ alkyl, (CH₂)₁₋₄-aryl, or (CH₂)₁₋₄—S(O)ₙ—C₁₋₄-alkyl;
R⁷ represents a seven to nine membered bicyclic heteroaryl or a seven to nine membered bicyclic heterocycloalkyl moiety, said moieties substituted with R⁸, R⁹ and R¹⁰;
R¹⁴ represents H; and
R¹¹ and R² along with the nitrogen and carbon atoms to which R¹¹ and R² are respectively attached can be taken together to form piperidine.

12. The compound of claim 11 wherein:
R⁴ represents C₁₋₅ alkyl; and
R⁷ represents a nine membered bicyclic heteroaryl or a nine membered bicyclic heterocycloalkyl moiety, said moieties substituted with R⁸, R⁹ and R¹⁰.

13. The compound of claim 12 wherein:
R⁴ represents C₂₋₄ alkyl;
R⁷ represents an indolyl, benzoxazolyl or a benzimidazolyl moiety; and
R⁸, R⁹ and R¹⁰, independently represent H or —C₁₋₄ alkyl.

14. The compound of claim 1 selected from the group consisting of:
1-[4-(4-Piperazin-1-yl-phenyl)-thiazol-2-yl]-piperidine-2-carboxylic acid[1-(benzooxazole-2-carbonyl)-butyl]-amide;
1-(4′-Piperazin-1-yl-biphenyl-3-yl)-piperidine-2-carboxylic acid [1-(benzooxazole-2-carbonyl)-butyl]-amide;
1-(4′-Pyridin-3-yl-biphenyl-4-yl)-piperidine-2-carboxylic acid[1-(benzooxazole-2-carbonyl)-butyl]-amide;
1-(4-{2-[4-(4-Methyl-piperazin-1-yl)-phenyl]-thiazol-4-yl }-phenyl)-piperidine-2-carboxylic acid [1-(benzooxazole-2-carbonyl)-butyl]-amide;
1-{4-[2-(4-Methyl-piperazin-1-yl)-thiazol-4-yl]-phenyl}-piperidine-2-carboxylic acid[1-(benzooxazole-2-carbonyl)-butyl]-amide;
1-{2-[4-(4-Methyl-piperazin-1-yl)-phenyl]-thiazol-4-yl}-piperidine-2-carboxylic acid[1-(benzooxazole-2-carbonyl)-butyl]-amide;
6′-(4-Pyridin-3-yl-piperazin-1-yl)-3,4,5,6-tetrahydro-2H—[1 ,3′]bipyridinyl-2-carboxylic acid[1-(benzooxazole-2-carbonyl)-butyl]-amide;
1-{4-[4-(4-Methyl-[1,4]diazepan-1-ylamino)-piperidin-1-yl]-phenyl}-piperidine-2-carboxylic acid[1-(benzooxazole-2-carbonyl)-butyl]-amide; and
1-{4-[6-(1-Methyl-piperidin-4-ylamino)-pyridin-3-yl]-thiophen-3-yl}-piperidine-2carboxylic acid[1-(benzooxazole-2-carbonyl)-butyl]-amide.

15. A method of treating a disease in an animal in which cysteine protease activity contributes to the pathology and/or symptomatology of the disease, which method comprises administering to the animal a therapeutically effective amount of a compound of claim 1 or an individual isomer or mixture of isomers thereof.

16. A pharmaceutical composition comprising a therapeutically effective amount of a compound of claim 1 or an individual isomer or mixture of isomers thereof in combination with a pharmaceutically acceptable excipient.

17. The composition of claim 16 which further comprises one or more active ingredient(s) selected from the group consisting of (i) a therapeutically effective amount of a bisphosphonic acid or acid ester thereof or a pharmaceutically acceptable salt thereof and (ii) a therapeutically effective amount of an estrogen receptor agonist or a pharmaceutically acceptable salt thereof.

18. The composition of claim 17 wherein the bisphosphonic acid is selected from the group consisting of 1,1-dichloromethylene-1,1-diphosphonic acid, 1-hydroxy-3-pyrrolidin-1-ylpropylidene-1,1-bisphosphonic acid, 1-hydroxyethylidene-1,1-diphosphonic acid, 1-hydroxy-3-(N-methyl-N-pentylamino)propylidene-1,1-bisphosphonic acid, 6-amino-i-hydroxyhexylidene-1,1-bisphosphonic acid, 3-(dimethylamino)-1-hydroxypropylidene-1,1-bisphosphonic acid, 3-amino-1-hydroxypropylidene-1,1-bisphosphonic acid, 2-pyrid-2-ylethylidene-1,1-bisphosphonic acid, 1-hydroxy-2-pyrid-3-ylethylidene-1,1-bisphosphonic acid, 4-chlorophenylthiomethylenebisphosphonic acid and 1-hydroxy-2-(1H-imidazol-1-yl)ethylidene-1,1-bisphosphonic acid or acid ester thereof or a pharmaceutically acceptable salt thereof.

19. The composition of claim 18 wherein the bisphosphonic acid is 1,1-dichloromethylene-1,1-diphosphonic acid or a pharmaceutically acceptable salt thereof.

20. The composition of claim 19 which comprises 1,1-dichloromethylene-1,1-diphosphonate monosodium trihydrate.

21. A method for treating a disease in an animal in which inhibition of a cysteine protease can prevent, inhibit or ameliorate the pathology and/or symptomatology of the disease, which method comprises administering to the animal a therapeutically effective amount of compound of claim 1 or individual isomer or mixture of isomers thereof; or a pharmaceutically acceptable salt thereof.

22. The method of claim 21 wherein the disease is osteoporosis.

23. The method of claim 22 wherein the animal is a human.

24. The method of claim 23 wherein the human is a post-menopausal woman.

25. The method of claim 24 wherein the cysteine protease is cathepsin K activity.
